(19) United States
(12) Patent Application Publication
(10) Pub. No.: US 2020/0046793 A1
(43) Pub. Date: Feb. 13, 2020

(54) ITCHING WATER APPLICABLE TO POSTOPERATIVE WOUND ITCHING AND A PROCESS FOR PREPARING IT

(71) Applicant: Yuankui Chen, Linyi (CN)

(72) Inventor: Yuankui Chen, Linyi (CN)

(21) Appl. No.: 16/114,232

(22) Filed: Aug. 28, 2018

(30) Foreign Application Priority Data
Aug. 10, 2018 (CN) 201810905732.3

(51) Int. Cl. A61K 36/83; A61K 36/258; A61K 36/40; A61K 36/744; A61K 36/48; A61K 36/328; A61K 36/732; A61K 36/539; A61K 36/758; A61K 36/537; A61K 36/232; A61K 36/708; A61K 36/888; A61K 36/324; A61K 36/54; A61K 45/06; A61K 47/10; A61K 9/08; A61P 17/04

(52) U.S. Cl. CPC A61K 36/83; A61P 17/04; A61K 36/40; A61K 36/744; A61K 36/48; A61K 36/328; A61K 36/732; A61K 36/539; A61K 36/758; A61K 36/537; A61K 36/232; A61K 36/708; A61K 36/888; A61K 36/324; A61K 36/54; A61K 45/06; A61K 47/10; A61K 9/08; A61K 36/258

(57) ABSTRACT

An itching water applicable to postoperative wound itching and a process for the production of it are provived. The itching water comprising Lignum Aquilariae Resinatum, Radix Notoginseng, Cornus Officinalis, Fructus Gardenia, Radix Flemingiae, Myrrh, Fructus Chaenomelis, Radix Scutellariae, Rhizoma Alismatis, Zanthoxylum Pipertum DC, Salvia Miltiorrhiza, Radix Angelicae Sinensis, Rheum Palmatum, Angelica Dahurica, Acorus tatarinowii, Radix Paeoniae Rubra, Lignum Sappan, Cinnamomum Cassia Presl, Radix Sanguisorbae, Asarum Sieboldii, Rhizoma Atractylodis, Pinellia Ternate, Common Clubmoss Herb, Pyritum, Radix Curcumae Aromaticae, Radix Saposhnikoviae, Rhizoma et Radix Notopterygii, Cortex Moutan, Boswellia Carteri, Radix Linderae and medical ethanol. The itching water of the invention has reasonable composition, less dosage, environment protection, simple operation, remarkable therapeutic effect and less side effects.

### BACKGROUND OF THE INVENTION

### 1. Field of the Invention

The invention relates to the medical field and in particular, relates to itching water applicable to postoperative wound itching and a process for preparing it.

### 2. Description of the Related Art

The incidence of postoperative wound itching is high but there is no specific treatment available for it at the present stage; why it is itchy when the wound is healed? Human skin is divided into multiple layers from the perspective of the skin structure, wherein the lowest layer of cells in the the epidermis is called the germinal layer, which has strong vitality and can continuously grow and reproduce. It will be itchy when the wound is large or the wound that goes deeply into the dermis is healed, because a new tissue called connective tissue is supplemented when a deeper wound is healed. The connective tissues are grown in the new blood vessels and nerves that are dense, and the itching sensation is thus caused. The regenerative capacity of various tissues in the human body is also varied, wherein the nerve tissue has the low regenerative capacity and appears late in wound healing. Generally, when the nerve is going to grow well, the wound is also going to grow well; then the new nerve endings and blood vessels have grown into the connective tissue, and the local consciousness recovers gradually, thus the wound itching occurs at this time.

There is no specific medicine available to treat wound itching at present, which makes the patient suffer from the wound itching, and seriously affects their physical and mental health and normal life.

Therefore, itching water applicable to postoperative wound itching is in desperately need.

### SUMMARY OF THE DISCLOSURE

The invention aims at overcoming the deficiencies of the prior art to the problems above, and providing itching water applicable to postoperative wound itching and a method of preparing thereof, which effectively relieves itching and accelerates the wound healing.

To achieve the objective above, the invention adopts the following technical solution:

Itching water applicable to postoperative wound itching, comprising Lignum Aquilariae Resinatum, Radix Notoginseng, Cornus officinalis, Fructus Gardenia, Radix Flemingiae, Myrrh, Fructus Chaenomelis, Radix Scutellariae, Rhizoma Alismatis, Zanthoxylum Pipertum DC, Salvia Miltiorrhiza, Radix Angelicae Sinensis, Rheum Palmatum, Angelica Dahurica, Acorns tatarinowii, Radix Paeoniae Rubra, Lignum Sappan, Cinnamomum Cassia Presl, Radix Sanguisorbae, Asarum Sieboldii, Rhizoma Atractylodis, Pinellia ternate, Common Clubmoss Herb, Pyritum, Radix Curcumae Aromaticae, Radix Saposhnikoviae, Rhizoma et Radix Notopterygii, Cortex Moutan, Boswellia Carteri, Radix Linderae and medical ethanol.

Preferably, the components have the preferable parts by weight as follows: 20-60 parts of Lignum Aquilariae Resinatum, 20-60 parts of Radix Notoginseng, 20-60 parts of Cornus Officinalis, 20-60 parts of Fructus Gardenia, 50-150 parts of Radix Flemingiae, 20-60 parts of Myrrh, 20-60 parts of Fructus Chaenomelis, 20-60 parts of Radix Scutellariae, 20-60 parts of Rhizoma Alismatis, 20-60 parts of Zanthoxylum Pipertum DC, 20-60 parts of Salvia Miltiorrhiza, 20-60 parts of Radix Angelicae Sinensis, 20-60 parts of Rheum Palmatum, 20-60 parts of Angelica Dahurica, 20-60 parts of Acorns Tatarinowii, 20-60 parts of Radix Paeoniae Rubra, 20-60 parts of Lignum Sappan, 20-60 parts of Cinnamomum Cassia Presl, 20-60 parts of Radix Sanguisorbae, 20-60 parts of Asarum Sieboldii, 20-60 parts of Rhizoma Atractylodis, 20-60 parts of Pinellia Ternate, 20-60 parts of Common Clubmoss Herb, 20-60 parts of Pyritum, 20-60 parts of Radix Curcumae Aromaticae, 20-60 parts of Radix Saposhnikoviae, 20-60 parts of Rhizoma et Radix Notopterygii, 20-60 parts of Cortex Moutan, 60-100 parts of Boswellia Carteri, and 50-90 parts of Radix Linderae.

According to another objective of the invention there is provided a process for preparing the itching water applicable to postoperative wound itching, comprising mixing Lignum Aquilariae Resinatum, Radix Notoginseng, Cornus Officinalis, Fructus Gardenia, Radix Flemingiae, Myrrh, Fructus Chaenomelis, Radix Scutellariae, Rhizoma Alismatis, Zanthoxylum Pipertum DC, Salvia Miltiorrhiza, Radix Angelicae Sinensis, Rheum Palmatum, Angelica Dahurica, Acorns Tatarinowii, Radix Paeoniae Rubra, Lignum Sappan, Cinnamomum Cassia Presl, Radix Sanguisorbae, Asarum sieboldii, Rhizoma Atractylodis, Pinellia Ternate, Common Clubmoss Herb, Pyritum, Radix Curcumae Aromaticae, Radix Saposhnikoviae, Rhizoma et Radix Notopterygii, Cortex Moutan, Boswellia Carteri and Radix Linderae uniformly according to the parts by weight in claim 2 after drying, fully grinding, and putting into 75% medical ethanol for soaking for 30 days, collecting soak solution, and bottling.

The invention has the advantageous effects that the itching water of the invention has reasonable composition, less dosage, environment protection, simple operation, remarkable therapeutic effect and less side effects.

### DETAILED DESCRIPTION

The invention is further elaborated below through detailed embodiment, but the invention is not limited merely to the embodiment below. It will be obvious to those skilled in the art that modifications, combinations or substitutions of the invention may be made within the scope of the invention or without departing from the content, spirit and scope of the invention, and the modifications, combinations or substitutions thereof all fall within the scope of the invention.

Itching water applicable to postoperative wound itching, comprising 20-60 parts of Lignum Aquilariae Resinatum, 20-60 parts of Radix Notoginseng, 20-60 parts of Cornus Officinalis, 20-60 parts of Fructus Gardenia, 50-150 parts of Radix Flemingiae, 20-60 parts of Myrrh, 20-60 parts of Fructus Chaenomelis, 20-60 parts of Radix Scutellariae, 20-60 parts of Rhizoma Alismatis, 20-60 parts of Zanthoxylum Pipertum DC, 20-60 parts of Salvia Miltiorrhiza, 20-60 parts of Radix Angelicae Sinensis, 20-60 parts of Rheum Palmatum, 20-60 parts of Angelica Dahurica, 20-60 parts of Acorns Tatarinowii, 20-60 parts of Radix Paeoniae Rubra, 20-60 parts of Lignum Sappan, 20-60 parts of Cinnamomum Cassia Presl, 20-60 parts of Radix Sanguisorbae, 20-60 parts of Asarum Sieboldii, 20-60 parts of Rhizoma Atractylodis, 20-60 parts of Pinellia Ternate, 20-60 parts of Common Clubmoss Herb, 20-60 parts of Pyritum, 20-60 parts of Radix Curcumae Aromaticae, 20-60 parts of Radix Saposhnikoviae, 20-60 parts of Rhizoma et Radix Notopterygii, 20-60 parts of Cortex Moutan, 60-100 parts of Boswellia Carteri, and 50-90 parts of Radix Linderae.

Mix Lignum Aquilariae Resinatum, Radix Notoginseng, Cornus Officinalis, Fructus Gardenia, Radix Flemingiae, Myrrh, Fructus Chaenomelis, Radix Scutellariae, Rhizoma Alismatis, Zanthoxylum Pipertum DC, Salvia Miltiorrhiza, Radix Angelicae Sinensis, Rheum Palmatum, Angelica Dahurica, Acorns Tatarinowii, Radix Paeoniae Rubra, Lignum Sappan, Cinnamomum Cassia Presl, Radix Sanguisorbae, Asarum Sieboldii, Rhizoma Atractylodis, Pinellia Ternate, Common Clubmoss Herb, Pyritum, Radix Curcumae Aromaticae, Radix Saposhnikoviae, Rhizoma et Radix Notopterygii, Cortex Moutan, Boswellia Carteri and Radix Linderae uniformly according to the parts by weight in claim 2 after drying, fully grind, and put into 75% medical ethanol for soaking for 30 days, collect soak solution, and bottling.

The itching relief is achieved by spraying the itching water on the itching area in use.

### 1. Efficacy Criteria

Healed: symptoms disappeared completely, not itchy at all

Effective: symptoms disappeared substantially, not itchy substantially

Invalid: no noticeable improvement in symptoms

### 2. Statistics Table of Efficacy Result

| | Number of | Average | Time of | Healed | Effective | Invalid | Cure |
|---|---|---|---|---|---|---|---|
| Items | People | Age | Using | Number | Number | Number | Number |
| Male | 105 | 51 | 30 days | 100 | 2 | 3 | 97.1% |
| Female | 95 | 50 | 30 days | 90 | 3 | 2 | 97.9% |

The above table shows that the itching water is effective in treating itch.

The above embodiment is merely the preferred embodiment of the invention, and is not intended to limit the scope of the invention, thus equivalent modifications made according to the claims of the invention all fall within the scope of the claims of the invention.

1. Itching water applicable to postoperative wound itching, comprising Lignum Aquilariae Resinatum, Radix Notoginseng, Cornus Officinalis, Fructus Gardenia, Radix Flemingiae, Myrrh, Fructus Chaenomelis, Radix Scutellariae, Rhizoma Alismatis, Zanthoxylum Pipertum DC, Salvia Miltiorrhiza, Radix Angelicae Sinensis, Rheum Palmatum, Angelica Dahurica, Acorns Tatarinowii, Radix Paeoniae Rubra, Lignum Sappan, Cinnamomum Cassia Presl, Radix Sanguisorbae, Asarum Sieboldii, Rhizoma Atractylodis, Pinellia Ternate, Common Clubmoss Herb, Pyritum, Radix Curcumae Aromaticae, Radix Saposhnikoviae, Rhizoma et Radix Notopterygii, Cortex Moutan, Boswellia Carteri, Radix Linderae and medical ethanol.

2. The itching water applicable to postoperative wound itching according to claim 1, wherein the components have the preferable parts by weight as follows:
20-60 parts of Lignum Aquilariae Resinatum, 20-60 parts of Radix Notoginseng, 20-60 parts of Cornus Officinalis, 20-60 parts of Fructus Gardenia, 50-150 parts of Radix Flemingiae, 20-60 parts of Myrrh, 20-60 parts of Fructus Chaenomelis, 20-60 parts of Radix Scutellariae, 20-60 parts of Rhizoma Alismatis, 20-60 parts of Zanthoxylum Pipertum DC, 20-60 parts of Salvia Miltiorrhiza, 20-60 parts of Radix Angelicae Sinensis, 20-60 parts of Rheum Palmatum, 20-60 parts of Angelica Dahurica, 20-60 parts of Acorns Tatarinowii, 20-60 parts of Radix Paeoniae Rubra, 20-60 parts of Lignum Sappan, 20-60 parts of Cinnamomum Cassia Presl, 20-60 parts of Radix Sanguisorbae, 20-60 parts of Asarum Sieboldii, 20-60 parts of Rhizoma Atractylodis, 20-60 parts of Pinellia Ternate, 20-60 parts of Common Clubmoss Herb, 20-60 parts of Pyritum, 20-60 parts of Radix Curcumae Aromaticae, 20-60 parts of Radix Saposhnikoviae, 20-60 parts of Rhizoma et Radix Notopterygii, 20-60 parts of Cortex Moutan, 60-100 parts of Boswellia Carteri, and 50-90 parts of Radix Linderae.

3. A process for preparing said itching water applicable to postoperative wound itching in claim 1, comprising mixing Lignum Aquilariae Resinatum, Radix Notoginseng, Cornus Officinalis, Fructus Gardenia, Radix Flemingiae, Myrrh, Fructus Chaenomelis, Radix Scutellariae, Rhizoma Alismatis, Zanthoxylum Pipertum DC, Salvia Miltiorrhiza, Radix Angelicae Sinensis, Rheum Palmatum, Angelica Dahurica, Acorns Tatarinowii, Radix Paeoniae Rubra, Lignum Sappan, Cinnamomum Cassia Presl, Radix Sanguisorbae, Asarum Sieboldii, Rhizoma Atractylodis, Pinellia Ternate, Common Clubmoss Herb, Pyritum, Radix Curcumae Aromaticae, Radix Saposhnikoviae, Rhizoma et Radix Notopterygii, Cortex Moutan, Boswellia Carteri and Radix Linderae uniformly according to the parts by weight in claim 2 after drying, fully grinding, and putting into 75% medical ethanol for soaking for 30 days, collecting soak solution, and bottling.
